(19) United States
(12) Patent Application Publication
(10) Pub. No.: US 2023/0372633 A1
(43) Pub. Date: Nov. 23, 2023

(54) Brake Tower with Elastomeric Component and Medical Injection Device Including the Same

(71) Applicant: Becton, Dickinson and Company, Franklin Lakes, NJ (US)

(72) Inventor: Mokhtar Toorneini, Swindon (GB)

(21) Appl. No.: 18/027,641

(22) PCT Filed: Sep. 23, 2021
(86) PCT No.: PCT/US21/51715

§ 371 Date: Mar. 22, 2023

Related U.S. Application Data
(60) Provisional application No. 63/083,622, filed on Sep. 25, 2020.

(51) Int. Cl. A61M 5/315

(52) U.S. Cl. CPC A61M 5/31585; A61M 2205/0216

(57) ABSTRACT

Provided herein is a brake tower for a medical injection device, the brake tower having a proximal end, a distal end having a distal face, and a sidewall therebetween defining a longitudinal axis, and one or more elastomeric protrusions arranged on the distal face. Also provided herein is a medical injection device including a brake tower.

### CROSS-REFERENCE TO RELATED APPLICATION

The present application claims priority to U.S. Provisional Application Ser. No. 63/083,622, entitled “Brake Tower with Elastomeric Component and Medical Injection Device Including the same”, filed Sep. 25, 2020, the entire disclosure of which is hereby incorporated by reference in its entirety.

### BACKGROUND OF THE INVENTION

### Field of the Invention

The present disclosure relates generally to components useful as replacements for helical coil springs for medical devices, and, in particular embodiments or aspects, to injection devices including a brake tower having an elastomeric component.

### Description of Related Art

Medical injection devices often utilize resilient members, such as springs, placed between various components, to bias the components in order to ensure more accurate drug delivery. However, there are shortcomings to the use of current resilient members used in medical injection devices, such as a lack of control and load recovery. Accordingly, there is a need in the art for a more robust component to allow for greater load recovery.

### SUMMARY OF THE INVENTION

Provided herein is a brake tower for a medical injection device, the brake tower having a proximal end, a distal end having a distal face, and a sidewall therebetween defining a longitudinal axis. The brake tower further includes one or more elastomeric protrusions arranged on the distal face.

The one or more elastomeric protrusions may be formed from an elastomeric polymer. The one or more elastomeric protrusions may be formed from a rubber. The rubber may be a natural rubber or a synthetic rubber. The one or more elastomeric protrusions may be formed from a silicone. The silicone may be a room-temperature vulcanized silicone.

The proximal end, the distal end, and the sidewall may define a cylinder. The distal end may have a larger circumference than the proximal end and/or the sidewall. The sidewall may define an interior that is at least partially hollow. The distal face may include two to ten elastomeric protrusions arranged thereon. The one or more elastomeric protrusions may have a circular shape. The one or more protrusions may have a polygon shape.

Also provided herein is a medical injection device including a brake tower having a proximal end, a distal end having a distal face, and a sidewall therebetween defining a longitudinal axis, and one or more elastomeric protrusions arranged on the distal face.

### BRIEF DESCRIPTION OF THE DRAWINGS

FIG. 1 is a perspective view of a non-limiting embodiment or aspect of a brake tower with an elastomeric component according to a non-limiting embodiment or aspect as described herein;

FIG. 2 is a partial cross-sectional view of a medical injection device including a brake tower according to a non-limiting embodiment or aspect as described herein, showing a pre-engagement position;

FIG. 3 is a partial cross-sectional view of a medical injection device including a brake tower according to a non-limiting embodiment or aspect as described herein, showing an engagement position;

FIG. 4 is an exploded view of a medical injection device including a brake tower with an elastomeric component according to a non-limiting embodiment or aspect as described herein; and

FIG. 5 is a cross-sectional view of a medical injection device including a brake tower with an elastomeric component according to a non-limiting embodiment or aspect as described herein.

### DESCRIPTION OF THE INVENTION

The use of numerical values in the various ranges specified in this application, unless expressly indicated otherwise, are stated as approximations as though the minimum and maximum values within the stated ranges are both preceded by the word “about”. As used herein, the term “about” means the stated value ±10%. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases.

The figures accompanying this application are representative in nature, and should not be construed as implying any particular scale or directionality, unless otherwise indicated. For purposes of the description hereinafter, the terms “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations and step sequences, except where expressly specified to the contrary. Hence, specific dimensions and other physical characteristics related to the embodiments disclosed herein are not to be considered as limiting.

Provided herein is a brake tower with an elastomeric component for use as a replacement for a spring in a medical device.

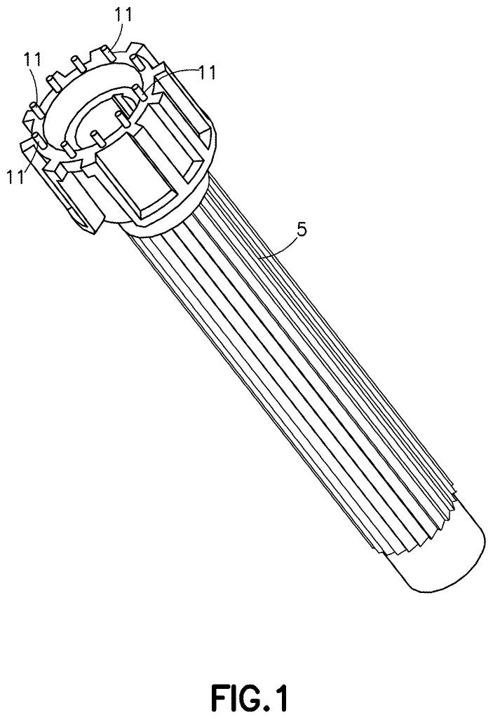

Turning to FIGS. 1-3, shown is a non-limiting embodiment or aspect of a brake tower 5 for a medical injection device. A brake tower can assume any useful configuration for use in a medical injection device. In non-limiting embodiments or aspects, brake tower 5 is cylindrical, and has a proximal end, a distal end, and a sidewall therebetween defining a longitudinal axis. In non-limiting embodiments or aspects, brake tower 5 is at least partially hollow, and the proximal end, distal end, and sidewall define an at least partially open interior that can receive one or more other components of a medical injection device, as will be described below. In non-limiting embodiments or aspects, interior of brake tower 5 includes one or more features for interacting, for example by reversible or irreversible locking, with a component received within the interior of the brake tower. For example, and without limitations, such features can include slots, tabs, recesses, and/or openings. Such features can, for example, prevent relative rotation between brake tower 5 and a component received within the brake tower interior, such as a brake tower core, a lead screw, and/or a piston rod.

In non-limiting embodiments or aspects, sidewall of brake tower 5 can include one or more splines or teeth configured to allow brake tower 5 to rotate when engaged by another component within a medical injection device, such as a setback member. In non-limiting embodiments or aspects, the one or more splines or teeth are configured to allow rotation of brake tower 5 in only a single direction.

In non-limiting embodiments or aspects, distal end of brake tower 5 is enlarged, for example by having a greater circumference than the proximal end. In non-limiting embodiments or aspects, distal end of brake tower 5 includes one or more features for interacting, for example by reversible or irreversible locking, with another component of a medical injection device, such as the housing. For example, and without limitations, such features can include one or more slots, tabs, recesses, and/or openings, and can prevent relative rotation between the brake tower and the component.

Brake tower 5 can be formed of any suitable material. In non-limiting embodiments or aspects, brake tower 5 is formed of a polymeric material, such as a plastic. In non-limiting embodiments or aspects, brake tower 5 is formed of a thermoplastic. In non-limiting embodiments or aspects, brake tower 5 is formed of a metal or a metal alloy.

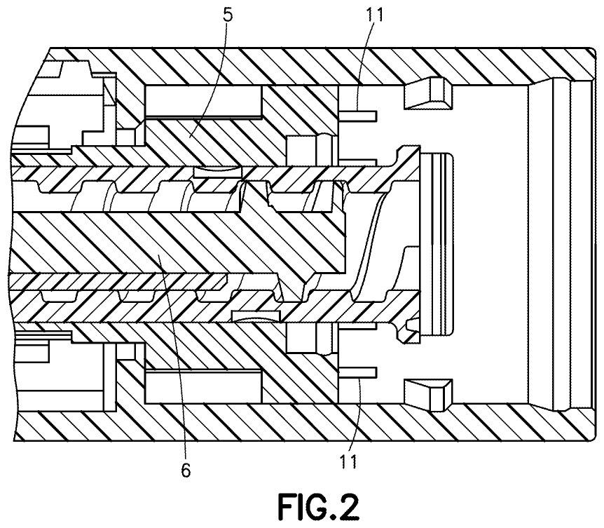

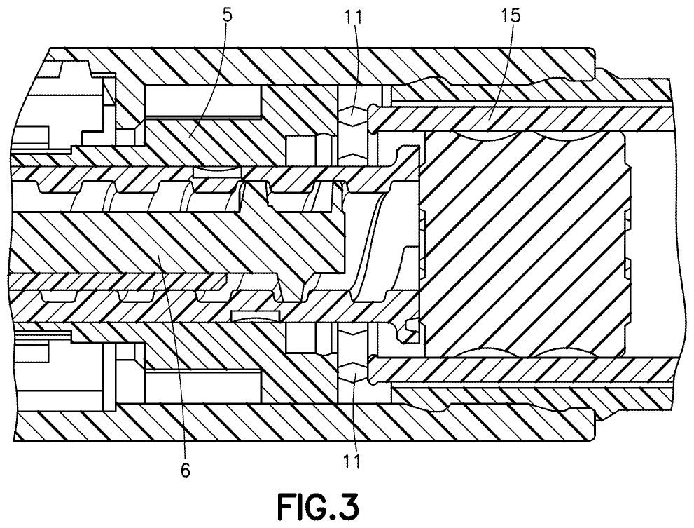

With continuing reference to FIGS. 1-3, distal end of brake tower 5 includes a distal face including one or more elastomeric protrusions 11 extending distally therefrom along the longitudinal axis. One or more elastomeric protrusions 11 serve to bias a cartridge received within a medical injection device, minimizing shifting of the components and thereby ensuring a more accurate delivery of a composition from the cartridge. As shown in FIG. 2, prior to assembly and insertion of the cartridge 15, the one or more elastomeric protrusions 11 have a first length and are uncompressed or unbiased. As shown in FIG. 3, after assembly and insertion of the cartridge 15, the cartridge 15 engages the one or more elastomeric protrusions 11 to compress and bias the one or more elastomeric protrusions 11 such that the one or more elastomeric protrusions 11 have a second length, which is smaller than the first length. In a non-limiting embodiment or aspect, distal face of brake tower 5 includes 2, 3, 4, 5, 6, 7, 8, 9, 10, or more elastomeric protrusions. In one non-limiting embodiment or aspect, distal face of brake tower 5 includes 5-10 protrusions.

One or more elastomeric protrusions 11 can be formed of any suitable elastomeric material, so long as the material provides the desired compressibility/expandability. In non-limiting embodiments or aspects, the elastomeric material is a highly resilient elastomeric material. In non-limiting embodiments or aspects, one or more elastomeric protrusions 11 are formed of a rubber. In non-limiting embodiments or aspects, the rubber is polyisoprene rubber, silicone rubber, and/or butyl rubber. In non-limiting embodiments or aspects, the rubber is butyl rubber (IIR), isoprene rubber (IR), butadiene rubber (BR), styrene-butadiene rubber (SBR), ethylene-propylene rubber (EPM), ethylene-propylene-diene rubber (EPDM), chlorosulphonated polyethylene (CSM), ethylene-vinyl acetate copolymer (EVA), styrene-isoprene rubber (SIR), thermoplastic elastomers, and/or natural rubbers. To the extent such rubbers are not adhesive by nature, one or more elastomeric protrusions 11 can be adhered to distal face of brake tower 5 by any suitable adhesive known to those of skill in the art.

In non-limiting embodiments or aspects, one or more elastomeric protrusions 11 are formed of a elastomeric copolymer, including, without limitation, thermoplastic elastomers, thermoplastic vulcanizates, styrene copolymers such as styrene-butadiene (SBR or SBS) copolymers, styrene-isoprene (SIS) block polymers or styrene-isoprene/butadiene (SIBS), in which the content of styrene in the styrene block copolymer ranges from about 10% to about 70%, and preferably from about 20% to about 50%. The elastomer composition can include, without limitation, antioxidants and/or inorganic reinforcing agents to preserve the stability of the elastomer composition, a vulcanizing agent, a vulcanizing accelerator, a vulcanizing activator, a processing aid, a filler, etc. to maintain and improve the physical properties and heat resistance of the rubber material. To the extent such copolymers are not adhesive by nature, one or more elastomeric protrusions 11 can be adhered to distal face of brake tower 5 by any suitable adhesive known to those of skill in the art.

In non-limiting embodiments or aspects, one or more elastomeric protrusions 11 are formed of a material having a Shore A value of 20-40 optionally 30-40, all values and subranges therebetween inclusive. In non-limiting embodiments or aspects, the one or more elastomeric protrusions 11 are formed of a material comprising an ethylene propylene diene monomer (EPDM) rubber. In non-limiting embodiments or aspects, one or more elastomeric protrusions 11 are formed of a material comprising an EPDM rubber, and various fillers/additives. In non-limiting embodiments or aspects, one or more elastomeric protrusions 11 are formed of an ultra-high molecular weight EPDM rubber (e.g., KELTAN 9565Q), various fillers (e.g., MISTRON Vapor), mineral oil, zinc oxide, stearic acid, antioxidant(s) (e.g., SONGNOX 1076), curing accelerator(s) (e.g., TBzTD), vulcanizing agent(s) (e.g., VULTAC 710), and sulfur (such as a wettable sulfur).

In non-limiting embodiments or aspects, one or more elastomeric protrusions 11 are formed of a foam. To the extent such foams are not adhesive by nature, one or more elastomeric protrusions 11 can be adhered to distal face of brake tower 5 by any suitable adhesive known to those of skill in the art.

In non-limiting embodiments or aspects, the elastomeric material is silicone. In non-limiting embodiments or aspects, the elastomeric silicone is a room-temperature vulcanized (RTV) silicone. RTV silicone is available commercially from a number of manufacturers, such as NuSil (Carpinteria, CA, USA). As RTV silicones can possess adhesive properties, in non-limiting embodiments or aspects, no additional adhesive need be applied to distal face of brake tower 5 to apply one or more elastomeric protrusions 11 thereto, though one of ordinary skill in the art can utilize additional adhesive materials as desired.

In non-limiting embodiments or aspects, one or more elastomeric protrusions 11 are formed of a mixture of materials, for example, and without limitation, a polymer, a rubber, a foam, and/or a silicone.

With continuing reference to FIG. 1, one or more elastomeric protrusions 11 can assume any useful shape, configuration, and/or orientation on distal face of brake tower 5. In non-limiting embodiments or aspects, one or more elastomeric protrusions have a circular shape or a polygon shape (e.g., a triangle, a quadrilateral, etc.). One or more elastomeric protrusions 11 can be oriented randomly on distal face of brake tower 5, or in a particular pattern.

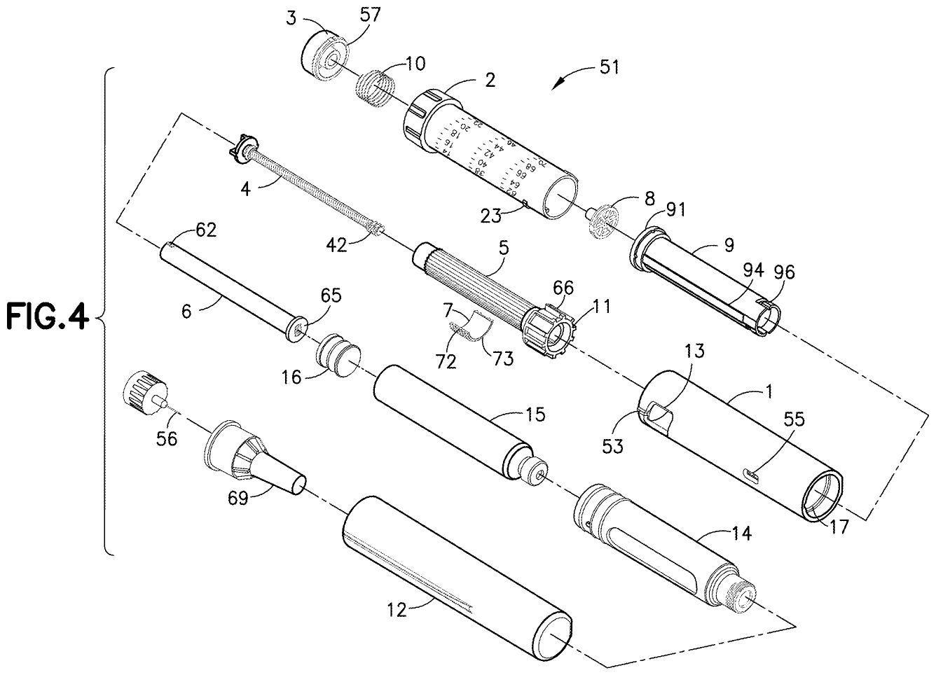

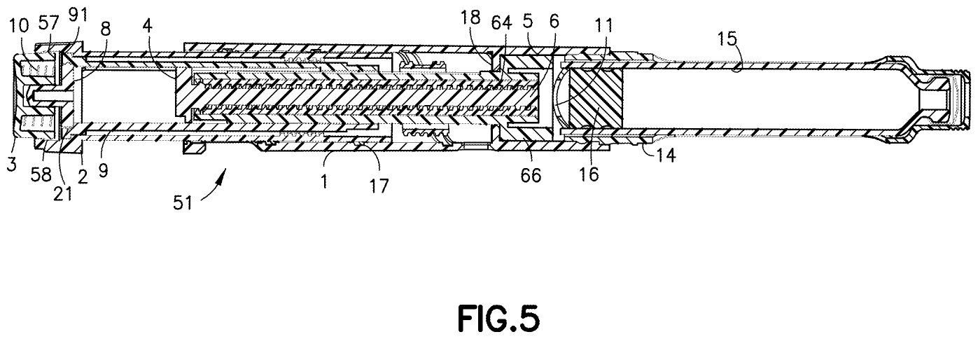

Also provided herein is a medical injection device including a brake tower with one or more elastomeric protrusions as described above. Such devices, such as injection pens, are described in U.S. Pat. No. 9,421,334, incorporated herein by reference in its entirety. With reference to FIGS. 4 and 5, shown are an exploded (FIG. 4) and cross-sectional (FIG. 5) view of an injection pen 51 for delivery of a composition to a user. As shown, injection pen 51 includes a pen upper body or housing 1, which houses a plurality of dose setting and injection components. Pen upper body 1 is connected to a cartridge housing 14, which houses cartridge 15. Injection pen 51 can also include a lower pen cap 12 to cover the cartridge 15 and cartridge housing 14 when injection pen 51 is not in use. As shown, injection pen 51 can include a dose set knob 2 that includes a knob-like portion that is rotated by a user to set a desired dose. Dose set knob 2 can also include a plurality of numerals, corresponding to a number of dosage units that is visible through a window 13 provided on pen upper body 1. A user rotates dose set knob 2 until the desired dose is visible in window 13. Pen upper body 1 can include an arrow or other indicator 53 to precisely indicate the set dose. Once the desired dose is set, a user presses a button 3 until the set dosage amount is completely injected. An outer shield 69 can cover a needle 56 to prevent accidental needle sticks upon removal of the lower pen cap 12.

Injection pen 51 can include push button 3, provided at a proximal end, closest to a user and farthest from needle 56, of pen upper body 1. Push button 3 can include an annular bead or rim 57 that engages with a corresponding annular groove (not shown) provided on the internal surface of dose set knob 2. The annular rim and groove connection can be a friction fit that maintains push button 3 in a biased position on dose set knob 2 under the force of a button spring 10, but allows push button 3 to be pushed into dose set knob 2 for injecting a set dose. The interior of push button 3 can accommodate a setback bearing insert 8 that rests on an internal surface at a proximal end of a setback member or driver 9. Push button 3 can be designed to rotate freely on setback bearing insert 8.

Setback member or driver 9 can be a cylindrical member, coaxial with and surrounded by dose set knob 2. Setback member 9 can be provided co-axially around brake tower 5 as described above. Brake tower 5 can be axially and rotatably fixed to pen upper body 1. In non-limiting embodiments or aspects, brake tower 5 co-axially surrounds a piston rod 6. Piston rod 6 can include a set of keys (not shown) that engage a slot (not shown) internal to brake tower 5 (as described above) to rotatably lock piston rod 6 to brake tower 5. Piston rod 6 can include a plurality of threads (not shown) provided on the interior surface thereof. Piston rod 6 can co-axially surround a lead screw 4 that includes a series of threads 42 at least at its distal end. Lead screw threads 42 can be configured to be in threaded engagement with the internal threads (not shown) provided on the interior of piston rod 6. Due to its threaded engagement with lead screw 4, piston rod 6 can be moved into cartridge 15 during injection to press on a stopper 16 provided inside cartridge 15 to expel a dose of medication.

With reference to the present disclosure, injection pen 51 includes brake tower 5 as described above. Brake tower 5 includes a distal face including one or more elastomeric protrusions as described above, positioned between brake tower 5 and cartridge 15 to bias cartridge 15 in a distal direction to prevent any movement of cartridge 15 during injection, and thus ensuring that an accurate dose is injected.

Following assembly, and when injection pen 51 is ready to be used, a dose for drug delivery can be set. To set a dose using injection pen 51 as described herein, a user rotates the knob portion of dose set knob 2 relative to pen upper body 1. An outer surface 59 of the dose set knob 2 can include a thread 23, which is in threaded engagement with a plurality of threads 17 provided on the internal surface of the pen upper body 1, as shown in FIGS. 4 and 5. Accordingly, as dose set knob 2 is rotated relative to pen upper body 1, dose set knob 2 screws or advances a distance out of pen upper body 1, as shown in FIG. 5. Dose set knob 2 can include an annular shoulder or rim 21 on the interior surface thereof near the proximal end, as shown in FIG. 5. This annular shoulder 21 can engage with an enlarged portion or head 91 of setback member 9, as shown in FIG. 5. Annular shoulder 21 of dose set knob 2 can include a series of teeth or ridges 22 that engage with a plurality of similarly shaped teeth or ridges (not shown) provided on enlarged head 91 of setback member 9. Dose set knob teeth 22 and setback member teeth can extend in opposite axial directions. During dose setting, dose set knob 2 can be free to rotate with respect to setback member 9 in both clockwise and counter-clockwise directions. As this occurs, the plurality of teeth or ridges 22 on dose set knob 2 can slip past the teeth provided on head portion 91 of setback member 9, thus providing a tactile signal or clicking noise to indicate the setting of a dosage amount.

With regard to the injection mechanism, as described above, lead screw 4 can include a plurality of threads 42 at its distal end that are in threaded engagement with a plurality of threads 64 that can be provided along the entire length of a hollow piston rod 6 as shown in FIG. 4. Piston rod 6 can be held non-rotatably with respect to pen upper body 1 due to a non-rotatable coupling with brake tower 5, which can be held axially and rotatably fixed with respect to the pen upper body 1. Piston rod 6 can include a key or set of keys 62 at its distal end that engage with a slot (not shown) provided on the internal surface of the brake tower 5 to prevent relative rotation therebetween while permitting piston rod 6 to move axially with respect thereto. Threads 42 of lead screw 4 can include a flat portion (not shown) corresponding to a flat portion 65 of piston rod 6, such that axial movement of the lead screw during dose setting does not result in axial movement of the piston rod 6. Accordingly, rotation of lead screw 4 during injection of a dose can cause the threads 42 of lead screw 4 to engage threads 64 of the piston rod 6, thereby axially moving piston rod 6. Because piston rod 6 can be non-rotatable with respect to body 1, as lead screw 4 is caused to rotate during injection, as described above due to its rotational coupling setback member 9, piston rod 6 through its threaded engagement with lead screw 4 can be caused to move in the distal direction to press against stopper 16 provided in cartridge 15, thus expelling a liquid medication therefrom.

Although the devices have been described in detail for the purpose of illustration based on what is currently considered to be the most practical and preferred embodiments, it is to be understood that such detail is solely for that purpose and that the systems and methods are not limited to the disclosed embodiments, but on the contrary, is intended to cover modifications and equivalent arrangements that are within the spirit and scope of the appended claims. For example, it is to be understood that the present systems and methods contemplate that, to the extent possible, one or more features of any embodiment can be combined with one or more features of any other embodiment

1. A brake tower for a medical injection device, comprising:
a proximal end, a distal end having a distal face, and a sidewall therebetween defining a longitudinal axis; and
one or more elastomeric protrusions arranged on the distal face.

2. The brake tower according to claim 1, wherein the one or more elastomeric protrusions comprise an elastomeric polymer.

3. The brake tower according to claim 1, wherein the one or more elastomeric protrusions comprise a rubber.

4. The brake tower according to claim 3, wherein the rubber is a natural rubber.

5. The brake tower according to claim 3, wherein the rubber is a synthetic rubber.

6. The brake tower according to claim 1, wherein the one or more elastomeric protrusions comprise a silicone.

7. The brake tower according to claim 6, wherein the silicone is a room-temperature vulcanized silicone.

8. The brake tower according to claim 1, wherein the proximal end, the distal end, and the sidewall define a cylinder.

9. The brake tower according to claim 1, wherein the distal end has a larger circumference than the proximal end and/or the sidewall.

10. The brake tower according to claim 1, wherein the sidewall defines an interior that is at least partially hollow.

11. The brake tower according to claim 1, wherein the distal face comprises two to ten elastomeric protrusions arranged thereon.

12. The brake tower according to claim 1, wherein the one or more elastomeric protrusions comprise a circular shape.

13. The brake tower according to claim 1, wherein the one or more protrusions comprise a polygon shape.

14. A medical injection device comprising:
a housing having a distal end and a proximal end;
a brake tower comprising a proximal end, a distal end having a distal face, and a sidewall therebetween defining a longitudinal axis, and
one or more elastomeric protrusions arranged on the distal face;
a cartridge received within the housing distally of the brake tower and holding a composition therein;
a cartridge housing received within the housing distally of the brake member and configured to hold the cartridge therein;
an injection needle at a distal end of the housing and in fluid communication with the cartridge; and
an actuation member at a proximal end of the housing, the actuation member configured to actuate the medical injection device to deliver the composition through the injection needle,
wherein the one or more elastomeric protrusions arranged on the distal face of the brake tower bias the cartridge distally.
